(19) United States
(12) Patent Application Publication
(10) Pub. No.: US 2009/0324714 A1
(43) Pub. Date: Dec. 31, 2009

(54) DUAL ADHESIVE TECHNOLOGY

(75) Inventors: Xiuying LIU, Glen Rock, NJ (US); John KRESEVIC, New Windsor, NY (US)

Correspondence Address: Pepper Hamilton LLP/Wyeth, 400 Berwyn Park, 899 Cassatt Road, Berwyn, PA, 19312-1183, US

(73) Assignee: WYETH, Madison, NJ (US)

(21) Appl. No.: 12/492,480

(22) Filed: Jun. 26, 2009

Related U.S. Application Data
(60) Provisional application No. 61/076,385, filed on Jun. 27, 2008.

(51) Int. Cl. A61K 9/20; A61K 9/00; A61K 31/56; A61K 31/16; A61K 31/60; A61K 31/192; A61K 31/20; A61K 31/14; A61K 31/305; A61P 43/00

(52) U.S. Cl. 424/465; 424/484; 424/488; 424/486; 424/485; 514/182; 514/626; 514/165; 514/568; 514/629; 514/560; 514/643; 514/496; 514/177

(57) ABSTRACT

A dual adhesive layer dosage form for delivery of active agent to and across, the mucosa is disclosed. Particularly, bioadhesive tablets for administration at the vaginal mucosa are disclosed as having a central active layer sandwiched between two bioadhesive layers.

This application claims benefit of priority to U.S. provisional application No. 61/076,385 filed on Jun. 27, 2008, which is hereby incorporated by reference.

### FIELD OF INVENTION

The invention relates to a pharmaceutical formulation for delivering an active pharmaceutical agent. More particularly, some embodiments of the invention relate to a tablet formulation for delivering an active agent via contact with the body's mucosa.

### BACKGROUND OF THE INVENTION

Mucosa is moist tissue that lines some organs and body cavities throughout the body, including, for example, the nose, mouth, lungs, digestive tract, urethra, vagina, and rectum. Particularly, mucosa lines body passages that have contact with outside air. Glands along the mucosa release mucus, making it difficult for biological or synthetic materials to hold or adhere to the mucosa. Thus, one key to delivering active agents at and across the mucosa is bioadhesion.

### SUMMARY OF THE INVENTION

According to some embodiments, the invention provides a tablet formulation for administration of an active agent at or across the body's mucosa, particularly the vaginal mucosa.

In some embodiments, the invention provides a pharmaceutical dosage form comprising:

an active layer comprising a pharmaceutically effective amount of an active agent in a swellable hydrophilic matrix;

at least two bioadhesive layers, comprising a bioadhesive agent;

wherein said active layer is disposed between said at least two bioadhesive layers.

In some embodiments, the hydrophilic matrix is selected from hydroxypropyl methyl cellulose (HPMC), polyethylene oxide, hydroxypropyl cellulose (HPC), hydroxyethyl cellulose (HEC), methylcellulose (MC), polyvinylpyrrolidone (PVP), xanthan gum, and guar gum, etc., or combinations thereof

In some embodiments, the bioadhesive agent is selected from polyacrylic acid derivatives, cellulose derivatives, substances of natural origin, protein, mucilaginous substances from edible vegetables, and combinations thereof.

In some embodiments, the invention provides a pharmaceutical dosage form comprising:

an active layer comprising an effective amount of an active agent in a swellable hydrophilic matrix, wherein
said hydrophilic matrix is selected from hydroxypropyl methyl cellulose (HPMC), polyethylene oxide, hydroxypropyl cellulose (HPC), hydroxyethyl cellulose (HEC), methylcellulose (MC), polyvinylpyrrolidone (PVP), xanthan gum, and Guan Gum, etc., or combinations thereof,
said active agent is selected from: analgesics and/or anesthetics, anti-infective agents, spermicides, estrogens, progestin, deodorizers, or combinations thereof;

at least two bioadhesive layers, comprising a bioadhesive agent selected from polyacrylic acid derivatives, cellulose derivatives, substances of natural origin, protein, mucilaginous substances from edible vegetables, or a combination thereof;

wherein said active layer is disposed between said at least two bioadhesive layers.

In some embodiments, the hydrophilic matrix comprises about 10% to about 55% of said active layer by weight.

In some embodiments, the bioadhesive agent comprises about 10% to about 30% of said bioadhesive layers by weight.

In some embodiments, the bioadhesive agent is a polyacrylic acid derivative selected from polycarbophil, high molecular weight cross-linked acrylic acid polymers, polyamides, polycarbonates, polyalkylenes, polyalkyleneglycols, polyalkyleneoxides, polyalkyleneterephthalates, polyvinyl alcohols, polyvinyl ethers, polyvinyl esters, polyvinyl halides, polyglycolides, polysiloxanes, polyurethanes, or combinations thereof.

In some embodiments, the bioadhesive agent is a Carbomer Homopolymer Type B USP/NF.

In some embodiments, the active agent is selected from: Analgesics and/or anesthetics, Anti-infective agents, Spermicides, Estrogens, Progestin, deodorizers, or combinations thereof.

When the active agent includes an analgesic and/or anesthetic, it can be selected from aspirin, ibuprofen, COX-2 inhibitors and other non-steroidal anti-inflammatory drugs (NSAIDS), acetaminophen, opiates and morphinomimetics, lidocaine, prilocaine, or other analgesics and anesthetics known in the pharmaceutical arts, as well as combinations thereof. Those of skill in the art will readily appreciate the class of NSAID compounds includes, but is not limited to: salicylic acids such as aspirin (acetylsalicylic acid), choline magnesium trisalicylate, diflunisal, salsalate; propionic acids such asfenoprofen, flurbiprofen, ibuprofen, ketoprofen, naproxen, oxaprozin; acetic acids such as diclofenac, indomethacin, sulindac, tolmetin; enolic acids such asmeloxicam, piroxicam; fenamic acids such as meclofenamate, mefenamic acid; napthylalkanones such as nabumetone; pyranocarboxylic acids such asetodalac, pyrrolesketorolac; and COX-2 inhibitors such as celecoxib.

Suitable anti-infective agents include from antibiotics, sulfonamides, antivirals, antifungals, and antiprotozoan, or combinations thereof.

Suitable spermicidal active agents include nonoxynol-9, octoxynol-9, benzalkonium chloride, ricinoleic acid, and phenol mercuric acetates or combinations thereof.

Suitable estrogens include naturally-occurring and synthetic estrogesn, such as conjugated estrogens (CE), synthetic conjugated estrogens, esterified estrogens, 17β-estradiol, estradiol acetate, estropipate, and estradiol hemihydrate or combinations thereof.

In some embodiments, the estrogen is a conjugated estrogen (CE).

Suitable progestins include medroxyprogesterone acetate, norethindrone, norgestel, megestrol acetate, progesterone, levonorgestrel, drospirenone, norgestimate, methyltestosterone and combinations thereof.

In some embodiments, the bioadhesive layers are joined at their outer edges so as to substantially envelop the active layer.

In some embodiments, the invention provides a pharmaceutical tablet comprising:

an active layer comprising an effective amount of conjugated estrogens in a swellable hydroxypropylmethyl cellulose hydrophilic matrix;

two bioadhesive layers comprising Carbomer Homopolymer Type B USP/NF;

wherein said active layer is disposed between the two bioadhesive layers.

In some embodiments,
the active layer comprises about 8 to about 10% conjugated estrogens of said active layer by weight, and about 10% to about 55% hydroxypropylmethyl cellulose of said active layer by weight; and
the bioadhesive layers comprises about 20% Carbomer Homopolymer Type B USP/NF by weight of the bioadhesive layers.

### BRIEF DESCRIPTION OF THE DRAWINGS

FIG. 1 is a graph depicting the influence of levels of HPMC on CE release rate from single layer tablets at 1 and 2 hr time points after inverse transformation.

FIG. 2 is a graph depicting the influence of levels of HPMC on CE release rate from single layer tablets at 3 and 4 hr time points after inverse transformation

FIG. 3 is a graph depicting the influence of levels of HPMC on CE release rate from single layer tablets at 5 and 8 hr time points after inverse transformation

FIG. 4 is a cross-sectional view of a tablet in accordance with some embodiments of the invention.

FIG. 5 is a cross-sectional view of a tablet in accordance with some embodiments of the invention.

### DETAILED DESCRIPTION OF THE INVENTION

The invention involves a multi-layer pharmaceutical dosage form particularly suited to delivering one or more active agents at the mucosa. Generally, a dosage form is provided having an active agent center and exterior bioadhesive layers. The bioadhesive layers may be the same or different, and are adapted to adhere the dosage form to the mucosa, while the active agent is released. In this manner, delivery of active agent can be targeted and localized in areas where delivery has been difficult to achieve because of the slippery nature of the mucosa. Various embodiments of the invention may further employ controlled release agents, enteric coatings, absorption/permeability enhancers, and other pharmaceutically acceptable excipients.

In some embodiments, the pharmaceutical dosage form has three layers: a hydrophylic matrix swellable active agent containing layer sandwiched between two bioadhesive layers.

By sandwiched, it is meant that an active layer is located between two bioadhesive layers. That is, sequentially there is a first bioadhesive layer, an active layer, and a second bioadhesive layer.

In some embodiments, the pharmaceutical dosage form is a tablet.

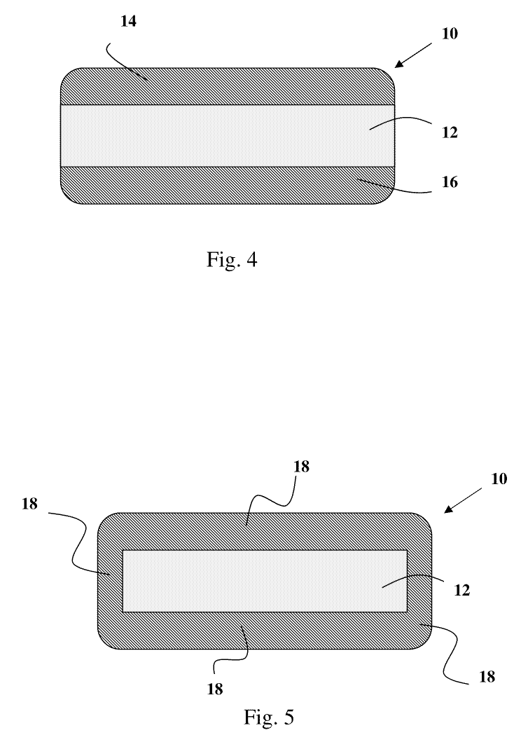

In some embodiments, exemplified in FIG. 4, the pharmaceutical dosage form 10 is a three layered tablet, wherein the center layer 12 has exposed edges. That is, at least a portion of the center active layer 12 is not covered by either of the bioadhesive layers 14, 16, or other excipient. In this arrangement, at least these exposed portions of active layer 12 are available for release directly, without potential interference from a bioadhesive layer.

In some embodiments, the pharmaceutical dosage form is a three-layered tablet 10, wherein the outer bioadhesive layers substantially envelop the center layer. In these embodiments, the outer edges of each bioadhesive layer are substantially connected, forming a continuous bioadhesive layer 18 surrounding substantially the entirety of the center active layer 12.

A variety of active agents or combination of active agents can be incorporated. Suitable active agents include, but are not limited to:

Analgesics and/or anesthetics such as aspirin, ibuprofen, COX-2 inhibitors and other non-steroidal anti-inflammatory drugs (NSAIDS), acetaminophen, opiates and morphinomimetics, lidocaine, prilocaine, or other analgesics and anesthetics known in the pharmaceutical arts, as well as combinations thereof. Those of skill in the art will readily appreciate the class of NSAID compounds includes, but is not limited to: salicylic acids such as aspirin (acetylsalicylic acid), choline magnesium trisalicylate, diflunisal, salsalate; propionic acids such asfenoprofen, flurbiprofen, ibuprofen, ketoprofen, naproxen, oxaprozin; acetic acids such as diclofenac, indomethacin, sulindac, tolmetin; enolic acids such asmeloxicam, piroxicam; fenamic acids such as meclofenamate, mefenamic acid; napthylalkanones such as nabumetone; pyranocarboxylic acids such asetodalac, pyrrolesketorolac; and COX-2 inhibitors such as celecoxib;

anti-infective agents such as antibiotics, sulfonamides, antivirals, antifungals, and antiprotozoan;

spermicides such as nonoxynol-9, octoxynol-9, benzalkonium chloride, ricinoleic acid, and phenol mercuric acetates;

hormones, such as but not limited to insulin, estrogens, and progestins;

natural and synthetic estrogens, such as conjugated estrogens (CE), synthetic conjugated estrogens, esterified estrogens, 17β-estradiol, estradiol acetate, estropipate, and estradiol hemihydrate;

estrogen receptor modulators, such as but not limited to Selective Estrogen Receptor Modulators (SERMS) such as afimoxifene (4-hydroxytamoxifen), arzoxifene, bazedoxifene, clomifene, femarelle (DT56a), lasofoxifene, ormeloxifene, raloxifene, tamoxifen, and toremifene;

progestins, such as medroxyprogesterone acetate, norethindrone, norgestel, megestrol acetate, progesterone, levonorgestrel, drospirenone, norgestimate, and methyltestosterone;

biological or biotherapuetic active agents such as proteins or peptides or portions thereof including, but not limited to, hormones, enzymes, proteases, kinases, receptor specific ligands, modulators, activators and inhibitors, wherein the biological actives may be natural or the result of bio- or genetic engineering;

absorption or permeation enhancers;

deodorizers;

and combinations of any of the above.

Other suitable active agents or combinations will be apparent to those of skill in the art. Those of skill in the art will recognize that the active agent can be used in any pharmaceutically acceptable form such as the free-base or a pharmaceutically acceptable salt.

The amount of active agent used will depend upon its pharmaceutically effective dose and delivery properties. Although it is possible that altering the bioadhesive content could affect dissolution profiles, the studies herein reveal that altering the amount of hydrophilic matrix (e.g. hydroxypropyl methyl cellulose) in the active layer has a rate controlling affect. Thus, the release rate of active agent can be adjusted by altering the level and or type of hydrophilic matrix system employed.

The content of the active agent(s) will vary according to the agent itself, and desired dosages. The use of conjugated estrogens (CE) is exemplified herein. Particularly, about 10.49 mg (8.74% by weight) CE dessication with lactose was used. Other CE formulations could be used, as well as other active agents, as described above. Other active agents may also be incorporated, making suitable adjustments in relative amounts depending on the desired properties.

Although the examples focus on delivery of CE, the amount and type of active ingredients is not limited to CE or estrogens, and may encompass additional active agents or combination of agents, without deviating from the scope and spirit of the invention. CE are used for estrogen replacement therapy (ERT), which can be beneficial for symptomatic relief of hot flushes, genital atrophy, and for the prevention of postmenopausal osteoporosis.

The active layer further contains a hydrophilic matrix system, for example, hydroxypropyl methylcellulose (HPMC). In general, HPMC seems to provide many advantages as a hydrophilic matrix system since it has excellent processability characteristics and many grades to choose from for formulation flexibility. HPMC is a pH-independent material and the drug release rates from HPMC matrices are generally independent of processing variables such as compaction pressure, drug particle size, and the incorporation of a lubricant. Other polymers, such as polyethylene oxide, hydroxypropyl cellulose (HPC), hydroxyethyl cellulose (HEC), methylcellulose (MC), polyvinylpyrrolidone (PVP), xanthan gum, guar gum, etc., or combinations thereof can also be employed in addition to or separate from HPMC. The examples herein focus on various formulations encompassing several levels related to HPMC. In these studies, about 10 to about 55% HPMC by weight was used in the exemplary formulations. Specifically, formulations including 10%, 20%, 35%, 45% and 55% HPMC were used. Studies incorporating an experimental design package (Design Expert® 6.09 software) were carried out in order to evaluate the effect of level of HPMC on the dissolution rate of CE. The balance of the active layer will vary with application, active agent, desired release profile, etc. Those of skill in the art will readily recognize suitable pharmaceutical excipients, fillers, binders, lubricants, and the like.

The active layer may optionally further contain controlled release agents. By controlled release, it is meant any release pattern that is not immediate, including any or all of delayed release, sustained release, extended release, continuous release, timed release, pH-dependent release, etc. Suitable controlled release agents include but are not limited to biodegradable polymers, carbomers, carnauba wax, carrageenan, cellulose acetate, cellulose acetate phthalate with ethyl cellulose, ceratonia, cetyl alcohol, cetyl esters wax, chitosan, guar gum, hydroxypropyl cellulose, hypromellose acetate succinate, methacrylate copolymer, methylcellulose, microcrystalline wax, paraffin, polycarbophil, polymethacrylates, etc.

As noted in the examples below, it is important to realize that although the bioadhesive layer could play a role in the release rate of the active layer, it need not play an substantial role. In some embodiments, of the invention, the bioadhesive layer does not control the release rate of the active agent. Rather, the release rate is controlled by the components of the active layer, which are chosen based upon the desired release profile, the active agent(s), and the condition to be treated, among other things.

The bioadhesive layers are provided on either side of the active layer, to form a three layered dosage form. Bioadhesion (or mucoadhesion) is generally defined as the ability of a biological or synthetic material to adhere to a mucous membrane, resulting in adhesion of the material to the tissue for a prolonged or extended period of time. Bioadhesion may enhance drug bioavailability due to the longer period of time in which the bioadhesive dosage form is in contact with the absorbing tissue versus a standard dosage form, such as tablet, sphere, capsule or film. To adhere to a mucous membrane, interaction, intermixing and/or amalgamation between a material and mucus, which is a highly hydrated, viscous anionic hydrogel layer protecting the mucosa, is needed.

Suitable adhesives include, but are not limited to, polyacrylic acid derivatives, cellulose derivatives, substances of natural origin, protein, and mucilaginous substances from edible vegetables. Combinations of these adhesives can also be used for increasing the mucoadhesive properties.

Suitable polyacrylic acid derivatives include, but are not limited to, polycarbophil, high molecular weight cross-linked acrylic acid polymers, polyamides, polycarbonates, polyalkylenes, polyalkyleneglycols, polyalkyleneoxides, polyalkyleneterephthalates, polyvinyl alcohols, polyvinyl ethers, polyvinyl esters, polyvinyl halides, polyglycolides, polysiloxanes, polyurethanes, or combinations thereof.

The examples herein focus on specific high molecular weight cross-linked acrylic acid polymers. However, it will be recognized that other adhesives can be used, with their relative concentrations being adjusted to account for the desired bioadhesive properties and the properties imparted by a particular agent.

Particularly, carbomer (CARBOPOL® 974P) was utilized as the bioadhesive polymer in the bioadhesive layers. Carbomer polymers are very high molecular weight polymers of acrylic acid, crosslinked with polyalkenyl ethers or divinyl glycol 3. When exposed to a pH environment above 4-6, the polymers swell up to 1000 times their original volume (and ten times their original diameter) in water to form a gel, providing a large adhesive surface area for maximum contact with the mucin. Above their pKa of 6±0.5, the carboxylate groups on the polymer backbone ionize, resulting in repulsion between the anions and further increasing the swelling of the polymer. Crosslinked polymers do not dissolve in water, but form colloidal gel dispersions.

Carbomer Homopolymer Type B USP/NF(CARBOPOL® 974P) was introduced specifically for use in oral and mucoadhesive contact applications such as controlled release tablets, oral suspensions and bioadhesives. In addition, Carbopol 974P provides the thickening, suspending, and emulsification properties to high viscosity systems for topical applications.

Suitable cellulose derivatives include but are not limited to alkylcelluloses, hydroxyalkylcelluloses, cellulose ethers, cellulose esters, nitrocelluloses, etc.

Substances of natural origin including chitosans, guar gum, xanthan gum, carrageenan, pectin, sodium alginate, dextrans, lectins, aminated gelatin, aminated pectin, hyaluronic acid, inulin, etc. may also be used as bioadhesives.

Proteins such as zein, serum albumin, and collagen may also be used as bioadhesives.

The relative amount of the bioadhesive can vary according to desired properties. For example, the type and amount of bioadhesive agent or agents can affect both adhesion strength and release rate of active agent (in addition to other properties). The relative amount of bioadhesive agent will be selected in accordance with the effect of a particular bioadhesive on the desired properties. As noted elsewhere, the bioadhesive layer can be designed to have little or no affect on the release rate.

Generally, about 10% to 30% bioadhesive agent(s) by weight of the bioadhesive layer is used. The exemplary compounds use about 20% carbomer. The balance of the bioadhesive layer can vary by application, but could be chosen from suitable pharmaceutically acceptable excipients, fillers, and the like.

In some embodiments one or both of the bioadhesive layers and/or the active layer may also contain absorption or permeation enhancers. In some instances, these enhancers can work additively or synergistically with the active agent(s) in the formulation. In some instances, the enhancer works synergistically with the active agent(s) to produce a therapeutic effect. In other instances, the enhancers may act as a preparatory treatment, readying the mucosa for the active agent(s). These may be physical or chemical in nature. For example, an enhancer, particularly when present in the bioadhesive layer(s), can open or thin the mucosal lining to allow ease of absorption. Some enhancers, whether in the bioadhesive layer(s) or the active layer could act to open up avenues for drug acceptance by preparing sites for drug delivery; e.g., by preparing, priming and or opening receptor area along the mucosa lining for drug transportation, etc . . . Those of skill in the art will readily recognize appropriate uses of various enhancers.

As used herein, “absorption” refers to the the movement and uptake of substances (liquids and solutes) into cells or across tissues such as mucosa, skin, intestine etc., by way of diffusion, osmosis, active transport or other means. For example, chemical entities are thought of as being absorbed.

As used herein, “permeation” refers to the process of spreading through or penetrating. For example, the term is more applicable in the administration of biologics and biotherapeutics which often (but not always) are discussed in terms of penetrating or permeating the cell, rather than in terms of absorption.

Although we believe the two terms could be used nearly synonymously, we note the subtle distinction.

In effect, the enhancers increase the bioavailability of one or more active agents by either adding to the therapeutic effect directly, or by preparing the mucosa for delivery of the active agent. The use of such enhancers could apply to the various delivery routes, oral, buccal, vaginal, rectal, etc. Some such enhancers include, but are not limited to, sodium caprate, sodium glycocholate, sodium glycodeoxycholate, sodium lauryl sulfate, sodium taurocholate, sodium taurodeoxycholate, sodium taurodihydrofusidate or combinations thereof. The amount of enhancer employed will vary depending on the enhancer used, the active agent, and other factors. Generally, however, the enhancer(s), when present, will account for about 0.25% to about 15% of the formulation. In some embodiments, the enhancer(s) will account for about 0.25% to about 10% of the formulation. In further embodiments, the enhancer will account for about 1 % to about 3% of the formulation. The active layer may contain additional pharmaceutically acceptable materials and excipients, including binders, lubricants, fillers, etc.

In some embodiments, the pharmaceutical formulation, for example tablets, can be enterically coated. Enterically coated tablets can contain an enteric coating as a layer external to the bioadhesive layer(s). Such tablets are particularly useful in oral administration, where delivery to the small intestine is desired. The enteric coating protects the bioadhesive layer(s) in the acidic pH of the stomach, eroding in the relatively higher pH of the small intestine, to reveal the bioadhesive layer(s). Generally, enteric coatings do not dissolve below pH of about 4. In some embodiments, the enteric coating does not dissolve below pH of about 5. Upon exposure of the bioadhesive layer(s), the tablet is free to adhere to the mucosa of the small intestine, where delivery of active agent is desired. By choice and manipulation of the enteric coating, delivery can be facilitated along the digestive tract, from the small intestine to the colon, as desired. Exemplary enteric coatings agents include cellulose acetate phthalate, guar gum, hypromellose acetate succinate, hypromellose phthalate, polymethacrylates, polyvinyl acetate phthalate, shellac, white wax, zein, etc.

In some embodiments, oral dosage forms are also designed to remain intact in the stomach and upper intestine, but to release the active substance further along the gastrointestinal tract, e.g. in the large intestine or in the colon. In these formulations, the later delivery is achieved through manipulating the enteric coating. In some cases, the thickness of the coating, or it components determine the release pattern. In some embodiments, the coating can be pH dependent, and to facilitate delivery to the lower bowel, will only release at pH above that found in the small intestine. In some instances, the release will not occur below pH of about 5. In some instances, the release will not occur below pH of about 6. In some instances, the release will not occur below pH of about 7. The site specific delivery of drugs to the colon has implications in a number of therapeutic areas such as the local treatment of colonic diseases such as Crohn's disease, irritable bowel syndrome, ulcerative colitis, colon cancer, etc. Local delivery into the colon facilitates delivery of active agent which would otherwise be susceptible to hydrolysis or degradation in the upper G.I. tract. Many active agents, particularly proteins and peptides, are acid-labile and cannot withstand the hostile environment of the upper G.I. tract. Delivery directly to the colon, which has reduced amounts of digestive enzymes and a more favorable pH will increase the chance of such actives being absorbed. Such absorption is also greatly enhanced by the fact that the active agent is adhered to the mucosa via the bioadhesive layer(s). Aside from local treatment of the lower G.I. tract, delivery to the lower bowel can also be useful in delaying systemic absorption in diseases such as asthma, arthritis, inflammation, etc.

Local and systemic delivery is possible with dual layer bioadhesive dosage forms according to the invention. Where local delivery is required, the bioadhesive material ensures that the dosage form will be held in place at or near the desired delivery site. In this manner, active substance(s) can be delivered where they are needed, and need not travel through the bloodstream before reaching the desired location. In other instances, the bioadhesive nature ensures that the dosage form will stay in place while the active substance(s) are released at or near an appropriate site for take up. In this manner, it is more likely that the entire dose will be absorbed for systemic effect. Through the choice of active substance(s) and the excipient(s), the local or systemic nature of the formulation can be controlled as desired.

Dual layer bioadhesive formulations of the invention are suitable for administration at any of the body's mucosa, but are particularly well-suited for buccal, sublingual or vaginal administration.

Buccal administration and sublingual administration differ from oral administration in that the formulation is not swallowed as with oral administration. With buccal administration, a tablet is placed between the cheek and gum. Drugs administered sublingually are given below the tongue. In either case, when the active substance (s) of the pharmaceutical formulation comes in contact with the mucous membrane, or buccal mucosa, it diffuses through the mucosa. The active substance diffuses into the capillaries there and enters directly into the venous circulation. In contrast, substances taken orally are subjected to the hostile environment of the gastrointestinal tract, where they can be degraded, by stomach acid, bile, or enzymatic action. What drug survives is absorbed and passes to the liver, where it may be extensively altered; this is known as the “first pass” effect of drug metabolism. Due to these activities, the oral route may be unsuitable for certain substances. Regardless, buccal and sublingual administration are often faster, and require lower doses since such administrations are not subject to degradation or “first pass” metabolism.

Although sublingual administrations are often thought of as having immediate or burst release properties, this need not be the case. As used herein, sublingual merely refers to the location of administration, under the tongue, and may have any desired release pattern.

With a tablet administered buccally, one bioadhesive layer adheres to the gum, while the other adheres to the cheek. In this manner, good adhesion is achieved to allow consistent drug delivery. Prior to the invention, a patient would have to actively hold a buccal dose in place with their cheek muscles, often manipulating a loose or wandering dose with their tongue or even fingers. Such a situation is undesirable, particularly from the point of patient compliance, and may be unsanitary. The dual adhesive dosage form reduces the need to actively hold the tablet in place, regardless of the delivery site.

Dual layer bioadhesive formulations of the invention are well-suited for vaginal applications because this type of application will provide localized effects which is often beneficial in these applications. Oral, topical and transdermal dosage forms could be systemically absorbed. Therefore, they can have unwanted effects on other body parts. Vaginal creams often require a special applicator and are messy to administer. Then, after application, they tend to run. Vaginal rings will bring a non-degradable foreign body in the organism. They require insertion and removal manipulations.

The dual layer bioadhesive formulation of the invention, in some embodiments, is in the form of a tablet. Due to the bioadhesive nature, the tablet can adhere to the mucous membrane, ensuring long term retention and thus delivery of the active agent where needed.

Although, a tablet formulation is exemplified herein, other dosage forms could be made in accordance with the invention. As described below, aside from manufacturing concerns, the particular tableting technique does not appear to be important for the desired properties of the tablet. Dry or wet granulation can be employed, and can be chosen based upon manufacturing concerns of the product being made. The tablets can be made by compression, as is well-known in the art.

### EXAMPLES

### Materials and Methods

### Development and Evaluation on Single Layer CE Tablets

In order to evaluate the influence of level of HPMC related to the dissolution of CE a one-factor response surface experimental design study was employed. Five HPMC concentrations (10, 20, 35, 45, and 55%) were evaluated. Eight runs in all as listed in Table 1 were generated using Design Expert® 6.09 software.

TABLE 1
Composition of CE Single Layer Tablet Formulations Generated By
Statistical Design

| | Input/Tablet (mg) (% W/W) | | | | | | | |
|---|---|---|---|---|---|---|---|---|
| Ingredient | Run 1 | Run 2 | Run 3 | Run 4 | Run 5 | Run 6 | Run 7 | Run 8 |
| CE | 10.49 | 10.49 | 10.49 | 10.49 | 10.49 | 10.49 | 10.49 | 10.49 |
| Desiccation | (8.74%) | (8.74%) | (8.74%) | (8.74%) | (8.74%) | (8.74%) | (8.74%) | (8.74%) |
| w/ | | | | | | | | |
| Lactose | | | | | | | | |
| Lactose | 79.21 | 25.21 | 49.21 | 67.21 | 79.21 | 25.21 | 37.21 | 49.21 |
| Monohydrate, | (66.01%) | (21.01%) | (41.01%) | (56.01%) | (66.01%) | (21.01%) | (31.01%) | (41.01%) |
| Spray | | | | | | | | |
| Dried, NF | | | | | | | | |
| Microcrystalline | 18 | 18 | 18 | 18 | 18 | 18 | 18 | 18 |
| Cellulose, | (15%) | (15%) | (15%) | (15%) | (15%) | (15%) | (15%) | (15%) |
| NF, | | | | | | | | |
| (Avicel | | | | | | | | |
| PH 101) | | | | | | | | |
| HPMC | 12 | 66 | 42 | 24 | 12 | 66 | 54 | 42 |
| K100M | (10%) | (55%) | (35%) | (20%) | (10%) | (55%) | (45%) | (35%) |
| Premium | | | | | | | | |
| CR | | | | | | | | |
| Magnesium | 0.3 | 0.3 | 0.3 | 0.3 | 0.3 | 0.3 | 0.3 | 0.3 |
| Stearate, | (0.25%) | (0.25%) | (0.25%) | (0.25%) | (0.25%) | (0.25%) | (0.25%) | (0.25%) |
| NF | | | | | | | | |

HPMC K100M Premium CR grade was used. HPMC K100M Premium Controlled Release (CR) grade was selected based on its controlled release properties. HPMC Premium CR grade is specially produced, ultra-fine particle size material, which can ensure the rapid hydration and gel formation that is so desired. CE Desiccation with lactose (CEDL) was used as the model drug of choice although other drugs can be incorporated. CEDL is a mixture of lactose and CE.

For this study, CE granulations for single layer tablets were manufactured in two different processes, wet granulation and dry granulation. For formulations with 10, 20, and 35% HPMC, a wet granulation method was employed. For formulations with 45% and 55% HPMC, the wet granulation process yielded very hard granules due to the high levels of HPMC K100M in these two formulations. Therefore, there were difficulties to dry the wet granulations to a desired moisture level. A dry granulation method, roller compaction, was used.

Wet Granulation Manufacture Procedures for CE Granulations with 10, 20, or 35% HPMC K100M Premium Controlled Release (CR)

A CEDL at 42.9 mg/g mixture was granulated with all other ingredients using water in a high shear granulator followed by the procedures below for a batch size of approximately 1.5 kg:

1. Mix CEDL with Lactose Spray Dried, Avicel®, and HPMC in a Collette shear mixer for approximately 5 minutes with plow at approximately 430 rpm.

2. Granulate the Step #1 blend by initiating the addition of water with plow and chopper set on approximately 430 and 1800 rpm, respectively. Add all the water within approximately 4 minutes.

3. Continue granulation for total of approximately 7 minutes.

4. Dry the wet granulation in a fluid bed dryer at an inlet temperature set point of 60° C. to achieve a target granulation LOD of 2%. A variation of ±0.5% moisture content is acceptable.

5. Pass the dried granulation through a Model “M” Fitzmill equipped with a #2A plate, set at a high speed (4500-4600 rpm), and impact set forward.

6. Mix the granulation of Step 5 in a V-Blender for approximately 10 minutes at approximately 22 rpm.

7. Remove about 100 g of Step 6 blend for use in Step 8.

8. Add the Magnesium Stearate (MS) through a #20 screen, in approximately equal portions, to each side of the V-blender. After the MS addition, add the Step 7 blend, in approximately equal portions, to each side of V-Blender. Blend for approximately 3 minutes. The quantity of MS added must be adjusted on a per tablet basis based on the quantity of granulation to be blended.

9. Discharge the Step 8 lubricated granulation into a double-bagged polyethylene bag with a desiccant bag in between the bags.

Dry Granulation Manufacture Procedures for CE Granulations with 45, or 55% HPMC K100M Premium Controlled Release (CR)

The dry granulation was completed using Fitzpatrick Chilsonator IR 220 by procedures below for a batch size of approximately 1 kg:
1. Screen intra-granular excipients except MS through a #30 mesh screen.

Blend in a 4 Qt V-blender for approximately 15 minutes at about 22 rpm.
2. Add intra-granular MS into the blender and blend for another approximately 3 minutes at about 22 rpm.
3. Granulate step 2 blend using Fitzpatrick Chilsonator IR 220 at following parameters:
Roll Pressure: approximately 309 psi
Roll Force: approximately 2556 lb/in
Roll Speed: approximately 7 rpm
VFS: approximately 180 rpm
HFS: approximately 25 rpm
4. Mill the ribbon using a Quadro Comil 197S at about 20% motor speed using screen with about 1.575 mm opening.
5. Weigh the milled materials.
6. Blend the milled materials in the 4 Qt V-blender for approximately 5 minutes at about 22 rpm.
7. Calculate the quantity of extra-granular MS needed based on the yield.
8. Weigh the MS and add to the blender and blend for approximately 3 minutes at about 22 rpm.
   Compression of Single Layer CE Tablets The single layer CE tablet was compressed using a Korsch XL100 compression machine with ¼″ round convex tooling. The targeted tablet weight was 120 mg. The compression force was adjusted in order to get tablets within the targeted hardness range of 7-12 kp and thickness of 0.14-0.18 inches.

### Content Uniformity of CE

Content uniformity of CE was determined on a sample size of 10 CE single layer tablets using USP method.

### Weight Variation

Weight variation of 1 00CE single layer tablets was evaluated using the Mocon Automatic Balance Analysis tester.

### Development and Evaluation on Dual Adhesive Technology (DAT) Dosage Form

In order to investigate the effects of bioadhesive layers on the release of CE from the Dual Adhesive Technology (DAT) dosage form, CE DAT dosage form was manufactured. The DAT dosage form has two carbomer non-active layers disposed on opposite sides of the center CE layer, which has the same formulation compositions of above single layer CE tablets (Table 2). The composition and manufacturing procedures of bioadhesive layers are listed in Table 3.

TABLE 2
Composition and Manufacturing Procedures of CE Layer of Dual Adhesive
Technology Dosage Form Formulations

| | Input/Tablet (mg) mg | | | | | | | |
|---|---|---|---|---|---|---|---|---|
| | % W/W) | | | | | | | |
| Ingredients | Run A | Run B | Run C | Run D | Run E | Run F | Run G | Run H |
| CE Desiccation | 10.49 | 10.49 | 10.49 | 10.49 | 10.49 | 10.49 | 10.49 | 10.49 |
| w/ Lactose | (8.74%) | (8.74%) | (8.74%) | (8.74%) | (8.74%) | (8.74%) | (8.74%) | (8.74%) |
| Lactose | 79.21 | 25.21 | 49.21 | 67.21 | 79.21 | 25.21 | 37.21 | 49.21 |
| Monohydrate, | (66.01%) | (21.01%) | (41.01%) | (56.01%) | (66.01%) | (21.01%) | (31.01%) | (41.01%) |
| Fast Flow, NF | | | | | | | | |
| Microcrystalline | 18 | 18 | 18 | 18 | 18 | 18 | 18 | 18 |
| Cellulose, NF, | (15%) | (15%) | (15%) | (15%) | (15%) | (15%) | (15%) | (15%) |
| (Avicel ® PH | | | | | | | | |
| 200) | | | | | | | | |
| HPMC K100M | 12 | 66 | 42 | 24 | 12 | 66 | 54 | 42 |
| Premium CR | (10%) | (55%) | (35%) | (20%) | (10%) | (55%) | (45%) | (35%) |
| Magnesium | 0.3 | 0.3 | 0.3 | 0.3 | 0.3 | 0.3 | 0.3 | 0.3 |
| Stearate, NF | (0.25%) | (0.25%) | (0.25%) | (0.25%) | (0.25%) | (0.25%) | (0.25%) | (0.25%) |

Procedures:
1. Screen CE desiccation w/Lactose with Avicel ® PH 200 through a #25 mesh screen.
2. Transfer above mixture to a 0.5 Qt V-blender and blend for approximately 5 minutes at approximately 22 rpm.
3. Screen HPMC through the same screen and transfer to a 1 Qt V-blender.
4. Transfer Step 2 blend to the same blender of Step 3.
5. Screen Lactose monohydrate through the same screen of Step 3 and transfer to the blender in Step 3.
6. Blend for approximately 15 minutes at approximately 22 rpm.
7. Screen Mg stearate through the screen together with approximately 10 g of the blend of Step 6.
8. Add the step 7 materials to the blender and blend for approximately 3 minutes at 22 rpm.

TABLE 3
Composition and Manufacturing Procedures of Bioadhesive Layer of
Dual Adhesive Technology Dosage Form Formulations

| Ingredient | Input/tablet (mg) | W/W % |
|---|---|---|
| Carbomer (Carbopol ® | 10 | 20 |
| 974P) | | |
| Microcrystalline Cellulose, | 39.75 | 79.5 |
| NF, (Avicel ® PH 200) | | |
| Magnesium Stearate, NF | 0.25 | 0.5 |

1. Screen Carbopol ® 974P with Avicel ® PH 200 through a #25 mesh screen.
2. Transfer the mixture of Step 1 to a 1 Qt V-blender.
3. Blend for approximately 15 minutes at approximately 22 rpm.
4. Screen Magnesium Stearate through the screen together with approximately 10 g of the Step 3 blend. Transfer to the blender.
5. Blend for approximately 3 minutes at approximately 22 rpm.

The DAT dosage form was manually compressed using Carver Press with 0.412 ×0.225 ×0.034 inches caplet convex tooling. The die was filled with 50 mg of bioadhesive layer blend. The blend was tapped slightly. Then it was filled with 120 mg of CE blend. After tapped slightly, the die was filled with another 50 mg of bioadhesive layer blend. The compression force was adjusted in order to have the DAT dosage form within the targeted hardness range of 16-22 kp and thickness of 0.17-0.20 inches. Various desirable CE release profiles as well as suitable in vivo bioadhesivities can be achieved by varying the weights of the active layer as well as bioadhesive layers.

Dissolution of Conjugated Estrogens from Single Layer and Dual Adhesive Technology (DAT) Dosage Form

The dissolution of CE was determined using USP Apparatus 2, at 50 rpm in 900 mL of 0.02M Sodium Acetate Buffer, pH 4.5. Filtered samples of the dissolution medium were taken at specified time intervals. The release of the active was determined on a reversed phase high performance liquid chromatography (HPLC).

### Content Uniformity and Weight Variation of CE Single Layer Tablets

Table 4 shows the results for the content uniformity and weight variation for these single layer tablet formulations. The data indicate that the formulations and manufacture processes can yield CE single layer tablets with excellent weight variation as well as content uniformity. The results also show that the content uniformity as well as weight variation is not dependent on the granulation process. However, a higher level of HPMC in the tablet tends to increase the value of content uniformity as well as weight variation.

TABLE 4
Content Uniformity and Weight Variation of CE Single Layer Tablets

| | | | Content | Weight |
|---|---|---|---|---|
| | | Granulation | Uniformity | Variation |
| Batch # | HPMC % | Process | (%) | (%) |
| Run 1 | 10 | Wet Granulation | 2.28 | 1.22 |
| Run 2 | 55 | Roller | 2.85 | 2.04 |
| | | Compaction | | |
| Run 3 | 35 | Wet Granulation | 2.6 | 1.48 |
| Run 4 | 20 | Wet Granulation | 1.88 | 1.84 |
| Run 5 | 10 | Wet Granulation | 0.92 | 1.04 |
| Run 6 | 55 | Roller | 3.21 | 2.00 |
| | | Compaction | | |
| Run 7 | 45 | Roller | 3.48 | 1.43 |
| | | Compaction | | |
| Run 8 | 35 | Wet Granulation | 2.16 | 1.35 |
| Run 7a | 45 | Direct | 3.49 | 1.47 |
| | | Compression | | |
| Run 7b | 45 | Wet Granulation | 2.78 | 1.41 |

Dissolution Profiles of CE from CE Single Layer Tablets with 45% HPMC Manufactured Using Different Processes

In order to evaluate whether different granulation processes can cause any changes of dissolution profiles of CE from the single layer tablets, CE single layer tablets with 45% HPMC K100M CR were manufactured using direct compression, wet granulation, and roller compaction processes. Dissolution profiles of CE were determined and shown in Table 5. From the data it can be seen that there is no significant difference of CE dissolution rates among these three different processes.

TABLE 5
Dissolution Profiles of CE from CE Single Layer Tablets with 45%
HPMC Manufactured Using Different Processes

| | Run 7a | Run 7b | Run 7 |
|---|---|---|---|
| Time (hr) | Direct Compression | Wet Granulation | Roller Compaction |
| 0 | 0 | 0 | 0 |
| 1 | 17 ± 0.4 | 20 ± 1.1 | 18 ± 1.7 |
| 2 | 29 ± 0.7 | 31 ± 1.4 | 30 ± 1.2 |
| 3 | 39 ± 1.1 | 40 ± 1.6 | 40 ± 1.4 |
| 4 | 47 ± 2.0 | 49 ± 1.2 | 48 ± 1.7 |
| 5 | 56 ± 2.2 | 57 ± 1.0 | 56 ± 1.8 |
| 8 | 75 ± 2.2 | 76 ± 1.7 | 75 ± 0.5 |

Influence of Levels of HPMC on CE Dissolution from Single Layer Tablets

A one factor response surface experimental design study was employed to evaluate the influence of HPMC levels on CE dissolution from the single layer tablets. Table 6 display the dissolution results for all eight runs generated from Design Expert® 6.09 software.

TABLE 6
Dissolution Profiles of Conjugated Estrogen from Eight Formulations of
Single Layer Tablets Generated from Experimental Design

| | Percent Released at Different Time Intervals | | | | | | |
|---|---|---|---|---|---|---|---|
| Run # | 0 hr | 1 hr | 2 hr | 3 hr | 4 hr | 5 hr | 8 hr |
| Run 1 | 0 | 67 ± 6.4 | 85 ± 6.0 | 94 ± 4.1 | 99 ± 2.4 | 100 ± 3.1 | 101 ± 2.1 |
| (10% HPMC) | | | | | | | |
| Run 2 | 0 | 15 ± 1.0 | 24 ± 0.9 | 33 ± 1.1 | 41 ± 1.2 | 49 ± 1.1 | 67 ± 1.7 |
| (55% HPMC) | | | | | | | |
| Run 3 | 0 | 22 ± 1.4 | 36 ± 1.9 | 47 ± 2.2 | 58 ± 2.4 | 66 ± 3.2 | 85 ± 3.5 |
| (35% HPMC) | | | | | | | |
| Run 4 | 0 | 32 ± 2.5 | 51 ± 2.8 | 65 ± 2.7 | 76 ± 3.2 | 84 ± 3.8 | 98 ± 2.9 |
| (20% HPMC) | | | | | | | |
| Run 5 | 0 | 79 ± 8.3 | 93 ± 4.7 | 100 ± 2.8 | 101 ± 1.6 | 101 ± 1.6 | 100 ± 1.6 |
| (10% HPMC) | | | | | | | |
| Run 6 | 0 | 14 ± 1.3 | 24 ± 1.2 | 32 ± 1.6 | 40 ± 1.5 | 47 ± 1.8 | 65 ± 2.4 |
| (55% HPMC) | | | | | | | |
| Run 7 | 0 | 18 ± 1.7 | 30 ± 1.2 | 40 ± 1.4 | 48 ± 1.7 | 56 ± 1.8 | 75 ± 0.5 |
| (45% HPMC) | | | | | | | |
| Run 8 | 0 | 23 ± 1.5 | 37 ± 2.4 | 49 ± 2.7 | 58 ± 2.7 | 67 ± 3.1 | 85 ± 3.1 |
| (35% HPMC) | | | | | | | |

The released CE percentages at 1, 2, 3, 4,5, and 8 hr of all model formulations were treated by Design Expert® 6.09 software. Suitable models for these experiments include linear, quadratic, and cubic models. The best fitting mathematical model was selected based on the comparisons of several statistical parameters including the standard deviation (ST), the multiple correlation coefficient (R²), adjusted multiple correlation coefficient (adjusted R²), and the predicted residual sum of square (PRESS), provided by Design Expert® 6.09 software. Among these statistical parameters, PRESS indicates how well the model fits the data, and for the chosen model it should be small relative to the other models under consideration⁶.
Linear model: Y=b₁+b₂X
Quadratic model: Y=b₁+b₂X+b₃X²
Cubic model: Y=b₁+b₂X+b₃X²+b₄X³
Note: X: HPMC level in tablet

In order to evaluate the effect of HPMC levels in CE single layer tablets on the dissolution pattern of CE, the factors and response variables were related using polynomial equation with statistical analysis. In order to find the best-fit model, CE release was inversely transformed. As shown in Table 7, the approximations of response values (inverse CE release percentage) (Y⁻¹_{CE 1 h}, Y⁻¹_{CE 2 h}, Y⁻¹_{CE 3 h}, Y⁻¹_{CE 4 h}, Y⁻¹_{CE 5 h}, and Y⁻¹_{CE 8 h}) based on the cubic model were most suitable since it exhibits a low standard deviation (ST), high R-Square values, a low PRESS, and a reasonable agreement between predicted R² versus adjusted R².

TABLE 7
Optimal Regression Equation for Each Response Variable for CE
Dissolution from CE Single Layer Tablets After Inverse Transformation

| Model | Coefficient | Y⁻¹_{CE 1 h} | Y⁻¹_{CE 2 h} | Y⁻¹_{CE 3 h} | Y⁻¹_{CE 4 h} | Y⁻¹_{CE 5 h} | Y⁻¹_{CE 8 h} |
|---|---|---|---|---|---|---|---|
| Linear | Std. Dev. × 1000 | 2.4 | 1.0 | 0.6 | 0.5 | 0.5 | 0.6 |
| | R-Square | 0.9899 | 0.9943 | 0.9952 | 0.9928 | 0.9884 | 0.9381 |
| | Adjusted R-Square | 0.9882 | 0.9934 | 0.9944 | 0.9916 | 0.9864 | 0.9277 |
| | Predicted R-Square | 0.9824 | 0.9904 | 0.9918 | 0.9872 | 0.9793 | 0.8879 |
| | PRESS × 1000000 | 60.2 | 9.7 | 3.8 | 3.1 | 2.8 | 3.7 |
| Quadratic | Std. Dev. × 1000 | 2.6 | 1.0 | 0.6 | 0.3 | 0.2 | 0.2 |
| | R-Square | 0.99 | 0.9947 | 0.9964 | 0.9975 | 0.9983 | 0.9959 |
| | Adjusted R-Square | 0.986 | 0.9925 | 0.995 | 0.9965 | 0.9976 | 0.9943 |
| | Predicted R-Square | 0.9773 | 0.9884 | 0.9917 | 0.993 | 0.9948 | 0.9876 |
| | PRESS × 1000000 | 77.5 | 11.7 | 3.8 | 1.7 | 0.7 | 0.4 |
| Cubic | Std. Dev. × 1000 | 1.5 | 0.5 | 0.4 | 0.3 | 0.2 | 0.2 |
| | R-Square | 0.9972 | 0.9992 | 0.9988 | 0.9983 | 0.9983 | 0.9969 |
| | Adjusted R-Square | 0.9951 | 0.9986 | 0.9979 | 0.9971 | 0.997 | 0.9946 |
| | Predicted R-Square | 0.9906 | 0.9966 | 0.9949 | 0.9926 | 0.9925 | 0.9876 |
| | PRESS × 1000000 | 32.2 | 3.4 | 2.3 | 1.8 | 1.0 | 0.4 |

Table 8 displays model F-value, lack of fit F-value, and an adequate precision based on the cubic model. From these values it can be seen that for all time points, cubic is the most suitable model since the model F-value implies that the model is significant. In addition, the lack of fit F-value implies the lack of fit is not significant relative to the pure error. Non-significant lack of fit is ample since it indicates that the model fits. For a model the adequate precision measures the signal to noise ratio. A ratio greater than 4 is desirable. Table 8 shows that all the time points have the adequate precision greater than 4.

TABLE 8
Model F-Value, Lack of Fit F-Values and Adequate Precision
for Cubic Model For Single Layer Tablets

| | Model | Lack of Fit F- | Adequate |
|---|---|---|---|
| | F-Value | Value | Precision |
| CE % 1 h | 473.92 (S) | 0.009363 (NS) | 51 |
| CE % 2 h | 1616.15 (S) | 0.36 (NS) | 94 |
| CE % 3 h | 1121.82 (S) | 0.60 (NS) | 78 |
| CE % 4 h | 795.47 (S) | 0.52 (NS) | 65 |
| CE % 5 h | 786.55 (S) | 0.66 (NS) | 64 |
| CE % 8 h | 430.66 (S) | 0.11 (NS) | 46 |

Note:
S = significant;
NS = not significant

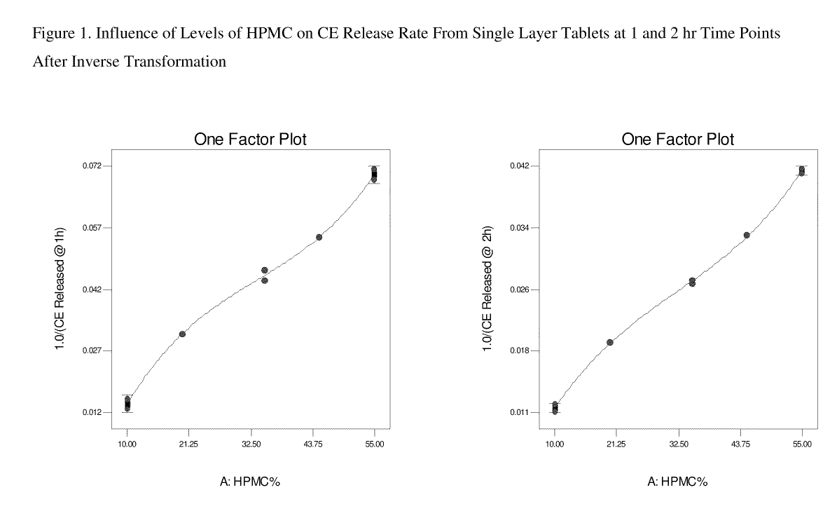

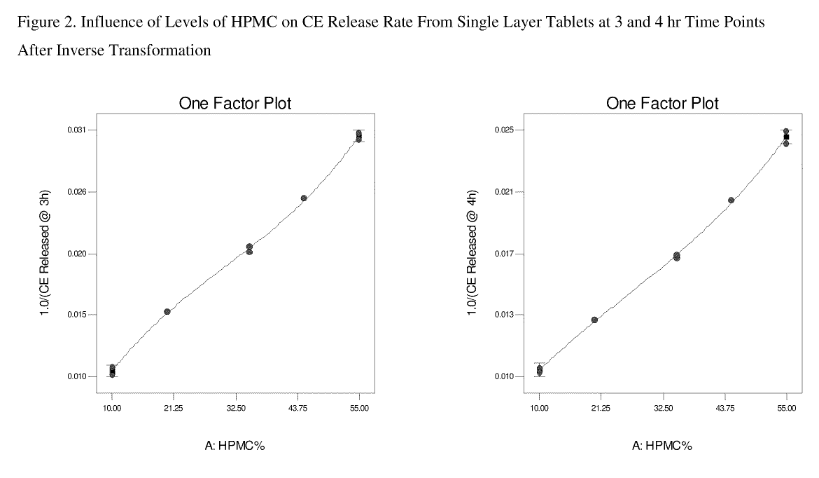

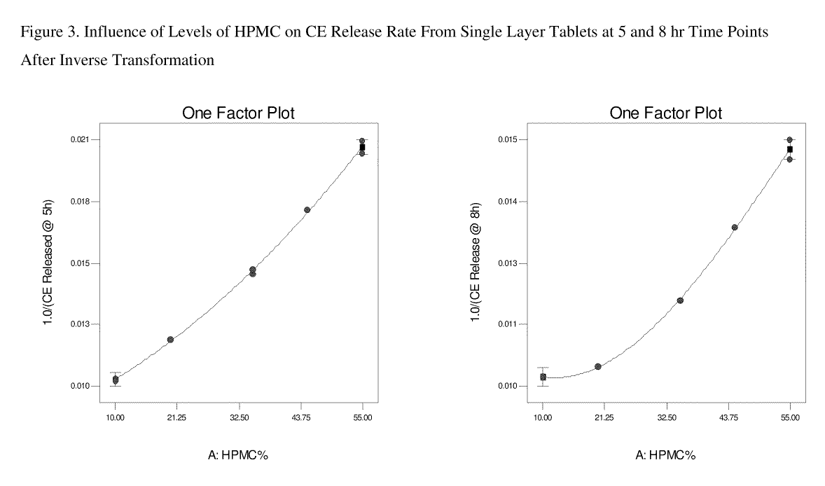

Table 9 lists all the coefficients for optimal regression equation for CE dissolution from the single layer tablets. FIGS. 1 to 3 illustrate the influence of levels of HPMC on the dissolution rate of CE. From these figures it can be concluded that HPMC has a negative impact on the CE dissolution rate.

TABLE 9
Optimal Regression Equation Coefficients for CE dissolution
From Single Layer Tablets

| | b₁ | b₂ | b₃ | b₄ |
|---|---|---|---|---|
| 1/CE Released @1 h | −0.015246 | 0.00367478 | −8.54672E−05 | 8.4924E−07 |
| 1/CE Released @2 h | −0.00181581 | 0.00162431 | −3.53327E−05 | 3.65582E−07 |
| 1/CE Released @3 h | 0.00309815 | 0.000871974 | −1.66372E−05 | 1.79919E−07 |
| 1/CE Released @4 h | 0.00628017 | 0.000423972 | −5.77692E−06 | 7.57885E−08 |
| 1/CE Released @5 h | 0.00857858 | 0.000125439 | 1.65288E−06 | 2.36745E−09 |
| 1/CE Released @8 h | 0.010623 | −0.000116391 | 5.31121E−06 | −3.09812E−08 |

Predicted Versus Actual Release of CE from Single Layer Tablets with 27.5% HPMC K100M CR

The dissolution rate of one batch of CE single layer tablets with 27.5% HPMC K100M CR was measured and compared with the predicted values based on the above cubic model equation. The dissolution profiles of this batch and the predicted profile are shown in Table 10.

TABLE 10
Comparison of Observed Dissolution profile with the Predicted value
of CE From Single Layer Tablets with 27.5% HPMC K100M CR

CE released (%)

| Time (hr) | Actual | Predict |
|---|---|---|
| 1 | 30 | 26 |
| 2 | 46 | 42 |
| 5 | 77 | 75 |
| 8 | 93 | 93 |

Difference factor (f1)⁷ and similarity factor (f2)⁸ were calculated to assess the similarity of these two profiles. Difference factor is a measure of the percent difference in the fractional dissolution between the two drug release curves over all time points:

$${f}_{1} = \frac{{\sum }_{t = 1}^{n} \vert {R}_{t} - {T}_{t} \vert}{\sum {R}_{t} } \times 100$$

Where Rₜ and Tₜ are the percent drug released from reference formulation and from the test formulation, respectively, at time t.

Similarity factor is function of the reciprocal of mean square-root transformation of the sum of squared error. It is a measure of the similarity in the fractional dissolution between the curves over all time points:

$${f}_{2} = 50 \times log \{ {[ 1 + \frac{1}{n} \sum {( {R}_{t} - {T}_{t} )}^{2} ]}^{- 0.5} \times 100 \}$$

where LOG=logarithm to base 10, n=number of sampling time points. FDA considers two dissolution profiles to be similar if f1 is between 0 and 15 and f2 is between 50 and 100. Also, the average difference at any dissolution sampling time point should not be greater than 15%. For this study, all the difference at any time point is less than 4%. The value of f1 is 4 and f2 is 76, respectively. Therefore, the release profile of the actual and the predicted are equivalent.
Release Mechanism of CE from Single Layer Tablets

It is believed that at the low level of HPMC (10%), CE release from the tablets follows a Fickian diffusion release (case I diffusional). As the level of HPMC increases, the CE release changes to a non-Fickian diffusion (0.45≦n≦0.89).

Influence of Levels of HPMC on CE Dissolution from Dual Adhesive Technology (DAT) Dosage Form

A one factor response surface experimental design study was employed to evaluate the influence of HPMC levels on CE dissolution from the DAT dosage form similar to that described above for the single layer formulation. For these eight formulations, the composition for the bioadhesive layer has the same level of Carbopol® 974P, 10%. The release rates of CE from these eight DAT dosage form formulations were measured. The results are displayed in Table 12 and FIG. 7.

TABLE 12
Dissolution Profiles of Conjugated Estrogen from Eight Formulations of
Dual Adhesive Technology Dosage Form Generated from Experimental Design

| | Percent Released at Different Time Intervals | | | | | | | |
|---|---|---|---|---|---|---|---|---|
| Run # | 0 hr | 2 hr | 3 hr | 4 hr | 5 hr | 6 hr | 8 hr | 12 hr |
| Run A | 0 | 46 ± 4.2 | 70 ± 4.4 | 94 ± 5.0 | 99 ± 6.1 | 99 ± 6.3 | 99 ± 6.7 | 99 ± 6.6 |
| Experiment 1 | | | | | | | | |
| (10% HPMC) | | | | | | | | |
| Run B | 0 | 19 ± 1.7 | 29 ± 1.4 | 39 ± 2.1 | 49 ± 2.3 | 59 ± 2.6 | 74 ± 2.7 | 91 ± 3.0 |
| Experiment 2 | | | | | | | | |
| (55% HPMC) | | | | | | | | |
| Run C | 0 | 23 ± 1.8 | 36 ± 1.6 | 49 ± 2.3 | 61 ± 1.6 | 71 ± 2.3 | 85 ± 2.3 | 97 ± 2.9 |
| Experiment 3 | | | | | | | | |
| (35% HPMC) | | | | | | | | |
| Run D | 0 | 38 ± 3.7 | 55 ± 4.3 | 73 ± 4.8 | 87 ± 4.8 | 95 ± 3.2 | 101 ± 1.5 | 102 ± 0.7 |
| Experiment 4 | | | | | | | | |
| (20% HPMC) | | | | | | | | |
| Run E | 0 | 46 ± 4.6 | 70 ± 4.5 | 92 ± 5.6 | 99 ± 2.4 | 98 ± 2.5 | 98 ± 2.3 | 98 ± 2.3 |
| Experiment 5 | | | | | | | | |
| (10% HPMC) | | | | | | | | |
| Run F | 0 | 18 ± 1.21 | 27 ± 2.0 | 37 ± 1.8 | 47 ± 1.6 | 56 ± 1.8 | 70 ± 1.8 | 88 ± 1.9 |
| Experiment 6 | | | | | | | | |
| (55% HPMC) | | | | | | | | |
| Run G | 0 | 21 ± 0.8 | 33 ± 1.2 | 44 ± 1.3 | 54 ± 1.9 | 64 ± 1.9 | 78 ± 1.4 | 93 ± 1.9 |
| Experiment 7 | | | | | | | | |
| (45% HPMC) | | | | | | | | |
| Run E | 0 | 23 ± 2.3 | 36 ± 2.3 | 49 ± 4.0 | 62 ± 3.8 | 72 ± 3.8 | 87 ± 3.9 | 99 ± 3.2 |
| Experiment 8 | | | | | | | | |
| (35% HPMC) | | | | | | | | |

The released CE percentages at 2, 3, 4, 5, 6, and 8 hr of all model formulations were treated by Design Expert® 6.09 software similarly to as described for the single layer tablet.

As shown in Table 12, at the initial time points, the influence of HPMC levels on the CE dissolution followed a quadratic model; however, at 6 and 8 hours time points, it changed to a cubic model, which was in agreement with the single layer tablet. This change might be due to the bioadhesive layer—(the Carbopol® 974P layer). As Carbopol® 974P is hydrated, it forms a gelatinous layer. This gel then acts as a rate-controlling membrane. At the initial stage of dissolution of these DAT dosage forms, CE is released either via diffusion from the surface gelatinous layer of Carbopol® 974P or the edge of the tablet, where CE penetrates through the HPMC gel matrix. At the same time, gels of Carbopol® 974P and HPMC also undergo erosion, which also promotes the CE release from the tablet. At the later stage of the dissolution most of the Carbopol® 974P gel dissolved. The only control CE release rate material was the HPMC gel. This could be the reason why the response values of Y_{CE 6 h} and Y_{CE 8 h} were fitted to a cubic model. The DAT dosage form behaved similarly to the single layer tablet, with respect to dissolution.

From these data it can be concluded that, like the single layer tablet, increase HPMC level has a negative impact on the CE dissolution rate.

From these data, it is seen that the DAT dosage form has a similar dissolution profile to the single layer tablet form. With the bioadhesive nature of the DAT formulation, delivery of the active agent is ensured since release rate is similar to that of non-adhesive formulations, but benefits from enhanced local delivery due to the ability of the bioadhesive to ensure prolonged or extended contact with the mucosa.

The Dual Adhesive Technology (DAT) dosage form formulation and its related manufacturing procedures are robust for producing a conjugated estrogens bioadhesive vaginal dosage form. Changing levels of HPMC in the center layer of the tablet can achieve different release rates of conjugated estrogens. The addition of the bioadhesive layers did not appear to negatively affect the dissolution profile. Addition of these layers should facilitate local delivery to the mucosa, by allowing greater and longer contact of the tablet, or other dosage form, with the mucosa.

The CE vaginal bioadhesive tablets described herein are for illustrative purposes only and are not meant to limit the invention in any way. As noted above, given the teachings herein, the tablet properties can be manipulated, particularly by altering the hydrophilic matrix system, and the relative amounts of adhesive agent in light of the properties associated with a given active agent or combination of active agents or desired tablet properties.

What is claimed is:

1. A pharmaceutical dosage form comprising:
an active layer comprising a pharmaceutically effective amount of an active agent in a swellable hydrophilic matrix;
at least two bioadhesive layers, comprising a bioadhesive agent;
wherein said active layer is disposed between said at least two bioadhesive layers.

2. The pharmaceutical dosage form of claim 1, wherein
said hydrophilic matrix is selected from hydroxypropyl methyl cellulose (HPMC), polyethylene oxide, hydroxypropyl cellulose (HPC), hydroxyethyl cellulose (HEC), methylcellulose (MC), polyvinylpyrrolidone (PVP), xanthan gum, and guar gum, or combinations thereof.

3. The pharmaceutical dosage form of claim 2, wherein said hydrophilic matrix comprises about 10% to about 55% of said active layer by weight.

4. The pharmaceutical dosage form of claim 1, wherein
said bioadhesive agent is selected from polyacrylic acid derivatives, cellulose derivatives, substances of natural origin, protein, mucilaginous substances from edible vegetables, and combinations thereof.

5. The pharmaceutical dosage form of claim 4, where said bioadhesive agent comprises about 10% to about 30% of said bioadhesive layers by weight.

6. The pharmaceutical dosage form of claim 4, wherein
said bioadhesive agent is a polyacrylic acid derivative selected from polycarbophil, high molecular weight cross-linked acrylic acid polymers, polyamides, polycarbonates, polyalkylenes, polyalkyleneglycols, polyalkyleneoxides, polyalkyleneterephthalates, polyvinyl alcohols, polyvinyl ethers, polyvinyl esters, polyvinyl halides, polyglycolides, polysiloxanes, polyurethanes, and combinations thereof.

7. The pharmaceutical dosage form of claim 6, wherein said bioadhesive agent is a Carbomer Homopolymer Type B USP/NF.

8. The pharmaceutical dosage form of claim 1, wherein said active agent is selected from: analgesics and/or anesthetics, anti-infective agents, spermicides, hormones, estrogens, estrogen receptor modulators, progestin, biological or biotherapuetic active agents, absorption or permeation enhancers, deodorizers and combinations thereof.

9. The pharmaceutical dosage form of claim 8, wherein said Analgesic and/or anesthetic is selected from non-steroidal anti-inflammatory drugs (NSAIDS), COX-2 inhibitors, opiates and morphinomimetics, lidocaine, prilocaine, and combinations thereof.

10. The pharmaceutical dosage form of claim 9, wherein said active agent is selected from aspirin, ibuprofen, acetaminophen and combinations thereof.

11. The pharmaceutical dosage form of claim 8, wherein said anti-infective agent is selected from antibiotics, sulfonamides, antivirals, antifungals, antiprotozoan, and combinations thereof.

12. The pharmaceutical dosage form of claim 8, wherein said spermicide is selected from nonoxynol-9, octoxynol-9, benzalkonium chloride, ricinoleic acid, phenol mercuric acetates and combinations thereof.

13. The pharmaceutical dosage form of claim 8, wherein said estrogens are selected from conjugated estrogens (CE), synthetic conjugated estrogens, esterified estrogens, 17β-estradiol, estradiol acetate, estropipate, estradiol hemihydrate and combinations thereof.

14. The pharmaceutical dosage form of claim 8, wherein said estrogens are conjugated estrogens (CE) or synthetic conjugated estrogens.

15. The pharmaceutical dosage form of claim 8, wherein said progestin is selected from medroxyprogesterone acetate, norethindrone, norgestel, megestrol acetate, progesterone, levonorgestrel, drospirenone, norgestimate, methyltestosterone and combinations thereof.

16. The pharmaceutical dosage form of claim 1, wherein said bioadhesive layers are joined so as to substantially envelop the active layer.

17. A pharmaceutical dosage form comprising:
an active layer comprising an effective amount of an active agent in a swellable hydrophilic matrix, wherein:
said hydrophilic matrix is selected from hydroxypropyl methyl cellulose (HPMC), polyethylene oxide, hydroxypropyl cellulose (HPC), hydroxyethyl cellulose (HEC), methylcellulose (MC), polyvinylpyrrolidone (PVP), xanthan gum, and guar gum, and combinations thereof;
said active agent is selected from analgesics and/or anesthetics, anti-infective agents, spermicides, estrogens, progestin, deodorizers, and combinations thereof
at least two bioadhesive layers, comprising a bioadhesive agent selected from polyacrylic acid derivatives, cellulose derivatives, substances of natural origin, protein, mucilaginous substances from edible vegetables, and combinations thereof;
wherein said active layer is disposed between said at least two bioadhesive layers.

18. The pharmaceutical dosage form of claim 17, wherein
said bioadhesive agent is a polyacrylic acid derivative selected from polycarbophil, high molecular weight cross-linked acrylic acid polymers, polyamides, polycarbonates, polyalkylenes, polyalkyleneglycols, polyalkyleneoxides, polyalkyleneterephthalates, polyvinyl alcohols, polyvinyl ethers, polyvinyl esters, polyvinyl halides, polyglycolides, polysiloxanes, polyurethanes, and combinations thereof.

19. The pharmaceutical dosage form of claim 18, wherein said bioadhesive agent is a Carbomer Homopolymer Type B USP/NF.

20. The pharmaceutical dosage form of claim 17, wherein said Analgesic and/or anesthetic is selected from non-steroidal anti-inflammatory drugs (NSAIDS), COX-2 inhibitors, opiates and morphinomimetics, lidocaine, prilocaine, or other analgesics and anesthetics known in the pharmaceutical arts, as well as combinations thereof.

21. The pharmaceutical dosage form of claim 20, wherein said active agent is selected from aspirin, ibuprofen, acetaminophen and combinations thereof.

22. The pharmaceutical dosage form of claim 17, wherein said Anti-infective agent is selected from antibiotics, sulfonamides, antivirals, antifungals, antiprotozoan, and combinations thereof.

23. The pharmaceutical dosage form of claim 17, wherein said spermicide is selected from nonoxynol-9, octoxynol-9, benzalkonium chloride, ricinoleic acid, phenol mercuric acetates and combinations thereof.

24. The pharmaceutical dosage form of claim 17, wherein said Estrogens are selected from conjugated estrogens (CE), synthetic conjugated estrogens, esterified estrogens, 17β-estradiol, estradiol acetate, estropipate, estradiol hemihydrate and combinations thereof.

25. The pharmaceutical dosage form of claim 17, wherein said estrogens are conjugated estrogens (CE) or synthetic conjugated estrogens.

26. The pharmaceutical dosage form of claim 17, wherein said Progestin is selected from medroxyprogesterone acetate, norethindrone, norgestel, megestrol acetate, progesterone, levonorgestrel, drospirenone, norgestimate, methyltestosterone and combinations thereof.

27. The pharmaceutical dosage form of claim 17, wherein said bioadhesive layers are joined so as to substantially envelop the active layer.

28. A tablet comprising
an active layer comprising an effective amount of conjugated estrogens in a swellable hydroxypropylmethyl cellulose hydrophilic matrix;
two bioadhesive layers comprising Carbomer Homopolymer Type B USP/NF;
wherein said active layer is sandwiched between said two bioadhesive layers.

29. The tablet of claim 27, wherein
said active layer comprises
about 8-10% conjugated estrogens of said active layer by weight;
about 10% to about 55% hydroxypropylmethyl cellulose of said active layer by weight; and
said bioadhesive layers comprises
about 20% Carbomer Homopolymer Type B USP/NF by weight of the bioadhesive layers.

30. The pharmaceutical dosage form of claim 1, wherein the active agent is a biological or biotherapeutical active agent.

31. The pharmaceutical dosage form of claim 1, further comprising:
an outer coating which dissolves at greater than about pH 4.

32. The pharmaceutical dosage form of claim 31, wherein said outer coating is selected to facilitate dissolution of the outer coating in the small intestine.

33. The pharmaceutical dosage form of claim 31, further comprising an outer coating which dissolves at greater than about pH 5.

34. The pharmaceutical dosage form of claim 33, wherein said outer coating is selected to facilitate dissolution of the outer coating in the lower bowel or colon.

35. The pharmaceutical dosage form of claim 1, wherein said active layer further comprises one or more controlled release agents.

36. The pharmaceutical dosage form of claim 1, wherein said active layer further comprises one or more absorption or permeation enhancer.

37. The pharmaceutical composition of claim 1, wherein at least one of said active layer and said bioadhesive layers further comprises one or more absorption/permeation enhancer.
